(19) United States
(12) Patent Application Publication
(10) Pub. No.: US 2024/0399050 A1
(43) Pub. Date: Dec. 5, 2024

(54) MEDICAL TUBE HUB

(71) Applicant: Danny W. Turpin, Heber City, UT (US)

(72) Inventor: Danny W. Turpin, Heber City, UT (US)

(21) Appl. No.: 18/680,531

(22) Filed: May 31, 2024

Related U.S. Application Data
(60) Provisional application No. 63/505,490, filed on Jun. 1, 2023.

(51) Int. Cl. A61M 5/14

(52) U.S. Cl. CPC A61M 5/1418

(57) ABSTRACT

A medical tubing hub can include: a body having a top, bottom, front and back. The body can include a plurality of conduits (e.g., 1, 2, 3, 4, 5, etc.) extending from a conduit top opening at the top to a conduit bottom opening at the bottom. The hub can include an attachment means configured for attaching the body to a patient. In some aspects, the dimension from the top to the bottom of the body (e.g., height) is larger than the dimension from the front to the back of the body (e.g., thickness). In some aspects, the attachment means is a clamp, clip, adhesive on the back, double sided adhesive tape, safety pin, magnet, magnetically responsive material, Velcro, cinch, or other attachment devices. In some aspects, the attachment means is a clamp, magnet, or magnetically responsive material.

### CROSS-REFERENCE TO RELATED APPLICATIONS

This patent application claims priority to U.S. Provisional Application No. 63/505,490 filed Jun. 1, 2023, which is incorporated herein by specific reference in its entirety.

### BACKGROUND

### Field

The present disclosure relates to a device configured to attach to a clothing item of a subject, in which the device is configured to retain and guide one or more medical tubes relative to the subject.

### Description of Related Art

In the current medical environment with patients having catheter tubing and/or oxygen tubes as well as other medical tubing that they have to wear, problems can occur during patient movement that dislodges a catheter or puts unwanted forces and strain on the medical tubing. For example, a single catheter tube may have a single site of entry into the patient's body, but that single catheter tube may be coupled with additional tubes, which makes it more likely that one of the medical tubes may snag or catch during patient movement. As a result, the catheter may have to be reset at the insertion point, along with additional wound care and possibly additional sterilization. The insertion point may need to be redressed because the stresses and strains that result from patient movement can cause the catheter to move in and out at the insertion point, which can allow for bacteria or other infectious agents to gain access to the compromised insertion point.

Additionally, while oxygen tubes can help supply oxygen to the patient, they restrict movement and often are allowed to dangle so that they can catch and be pulled out or away from the patient. Further problems occur when a patient has a catheter and oxygen tubing being used for a treatment or other health reasons.

Therefore, it would be advantageous to have a device or system that can be used in a method to inhibit medical tubing from being moved or compromise an insertion point.

### SUMMARY

In some embodiments, a medical tube hub can include: a body having a top, bottom, front and back. The body can include a plurality of conduits (e.g., 2, 3, 4, 5, etc.) extending from a conduit top opening at the top to a conduit bottom opening at the bottom. The hub can include an attachment means configured for attaching the body to a patient. In some aspects, the dimension from the top to the bottom of the body (e.g., height) is larger than the dimension from the front to the back of the body (e.g., thickness).

In some embodiments, each conduit includes a conduit slot that exposes the conduit at the front of the body. In some aspects, the medical tube hub can include at least two or at least five conduits. In some aspects, the conduit slot extends from the conduit top opening at the top to the conduit bottom opening at the bottom. In some aspects, the conduit has a lateral cross-sectional profile that is circular, oval, elliptical, triangular, square, rectangle, pentagon, hexagon, heptagon, octagon, polygon, or combinations thereof, the lateral cross-sectional profile being orthogonal with a longitudinal axis of the conduit. In some aspects, the conduit has a lateral cross-sectional profile that has a conduit cross-dimension (e.g., diameter for circular conduit) having a first width and the conduit slot has a slot cross-dimension from edge to edge that has a second width, wherein the first width is bigger than the second width, wherein the conduit cross-dimension and the slot cross-dimension edge to edge are both orthogonal to the longitudinal axis. In some aspects, the conduit with the conduit slot form an omega shape £2 cross-sectional profile with the front. In some aspects, the hub includes a separator between each conduit, wherein the separator extends from the top to the bottom. In some aspects, the body includes one less separator than the number of conduits. In some aspects, the plurality of conduits are parallel.

In some embodiments, the attachment means is oriented between the plurality of conduits and the back of the body. In some aspects, the attachment means is a clamp, clip, adhesive on the back, double sided adhesive tape, safety pin, magnet, magnetically responsive material, Velcro, cinch, or other attachment devices. In some aspects, the attachment means is a clamp, magnet, or magnetically responsive material. In some aspects, when Velcro or magnetic or magnetically responsive material is used, a corresponding fastening member can be provided that is removable from the attachment means and selectively couplable with the attachment means. The hub may include the removable fastening member attached thereto or a kit can be provided with the hub and removable fastening member.

In some embodiments, the attachment means includes a clamp. In some aspects, the clamp has a lever attached through a hinge to a receiver. In some aspects, the lever has a latch edge and the receiver has a receiver slot, wherein the latch edge and receiver slot are cooperatively configured such that when in a closed position the latch edge is received in the receiver slot. In some aspects, the lever and receiver are elongate. In some aspects, the lever and receiver extend between a first side to a second side of the body. In some aspects, the lever and receiver extend past the first side and past the second side of the body. In some aspects, the lever has a wedge shaped cross-sectional profile with a point at the latch edge. In some aspects, the receiver slot has a wedge shaped cross-sectional profile with a point at a trough of the receiver slot. In some aspects, the lever includes a fastening head opposite of the hinge. In some aspects, the fastening head includes a fastening tab adapted to fasten to the receiver. In some aspects, the receiver is devoid of a notch to receive the fastening tab, and thereby the fastening tab fastens with a fastening end of the receiver, the fastening end being opposite of the hinge. In some aspects, the receiver includes a notch to receive the fastening tab, and thereby the fastening tab fastens with the notch at the fastening end of the receiver, the fastening end being opposite of the hinge. In some aspects, the fastening tab is configured as a fastening hook. In some aspects, the fastening tab is configured as a fastening hook, wherein the fastening hook includes a hooking member such that there is a gap between the hooking member and the latch edge. In some aspects, the fastening end includes a bottom of the receiver that is cooperatively dimensioned with a dimension of the gap, such that the bottom of the receiver at the fastening end fits into the gap.

In some embodiments, the fastening means (e.g., clamp, magnet, Velcro) is coupled with the body of the hub. In some aspects, the fastening means is coupled with the body of the hub via an adhesive. In some aspects, the fastening means is integrated with the body of the hub. In some aspects, the fastening means includes at least one member that is integrated with the body so as to be a unitary body. In some aspects, the fastening means includes at least one member that is integrated with the body so as to be a unitary body made of the same material.

In some embodiments, the fastening means is a clamp that is coupled with the body of the hub, the body having a clamp recess that retains the clamp therein. In some aspects, the clamp recess is formed into the back of the body. In some aspects, the clamp recess is formed into the back and top of the body. In some aspects, the clamp recess is formed into the top of the body. In some aspects, the clamp recess is formed into the top of the body between the back and the front. In some aspects, the clamp recess is formed into the top of the body between the back and the front and positioned closer to the back. In some aspects, the clamp recess extends between the first end and second end. In some aspects, the clamp recess is formed into the top of the body between the back and the conduits.

In some embodiments, the lever of the clamp is longer than the receiver of the clamp. In some aspects, the lever is longer than the receiver such that the lever head extends past the fastener end when the clamp is in a closed position. In some aspects, the lever includes a grip member opposite of the lever edge. In some aspects, the fastener end includes a receiver head. In some aspects, the fastener end includes a receiver head that is bigger than a main body portion of the receiver. In some aspects, the clamp and clamp recess are orthogonal with the conduits.

In some embodiments, the body of the hub includes a back member located at the back. In some aspects, the body includes a back member located at the back, the back member cooperating with a main portion of the body to form the clamp recess.

In some embodiments, a kit can include the medical tube hub of one of the embodiments and one or more medical tubes that fit within the conduits. In some aspects, the one or more medical tubes are selected from oxygen tubes, catheter tubes, IV tubes, or other medical tubes. In some aspects, a kit can include the medical tube hub of one of the embodiments; and a clothing item having an attachment member.

In some embodiments, a kit can include the medical tube hub of one of the embodiments, wherein the attachment means is a clamp, clip, adhesive on the back, double sided adhesive tape, safety pin, magnet, magnetically responsive material, Velcro, cinch, or other attachment devices. The kit can also include a fastening mean that attaches to the attachment means, such as when the fastening means and attachment means are cooperatively at least one magnet, a magnet and magnetically responsive material, two sides of Velcro, or the like. In some aspects, the kit can include a clothing item having an attachment member (e.g., fastening means), wherein the attachment member is adapted to attach to the attachment means. In some aspects, the attachment member is a substrate to receive an adhesive, 2-sided adhesive tape, a magnetic substrate, a magnetically responsive substrate, a member having Velcro hooks, a member having Velcro loops, or combination thereof.

In some embodiments, a kit can include the medical tube hub of one of the embodiments and a clip having a clipping portion and a loop portion, in which the clipping portion is configured to attach to a clothing item and the loop portion is configured to receive a medical tube therein.

In some embodiments a kit can include the medical tube hub of one of the embodiments and a wrist band comprising one or more conduits configured to receive a medical tube therein.

In some embodiments, a method of retaining medical tubing to a subject can include: providing the medical tube hub of one of the embodiments; attaching the medical tube hub to the subject, such as via coupling with clothing worn by the subject; and attaching one or more medical tubes to the conduits of the medical tube hub. In some aspects, the one or more medical tubes are selected from oxygen tubes, catheter tubes, IV tubes, or other medical tubes.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIGS. 1A-1D include schematic drawings that show an embodiment of a medical tubing hub, which is configured in accordance with the descriptions provided herein.

FIG. 2 shows the medical tube hub retaining catheter/IV medical tubes and oxygen medical tubes.

FIG. 3A shows an article of clothing, configured as a medical gown, which can be used with the medical tubing hub and/or kit including the same.

FIG. 3B shows an embodiment of a medical tubing hub that can be adapted to be used with the article of clothing of FIG. 3A when having a member that fastens with the medical tubing hub.

FIGS. 4A and 4B show an embodiment of a medical tubing hub using a removable fastening member that can allow clothing to be received between the removable fastening member and the main body of the medical tubing hub and secured thereto.

FIG. 5A shows an embodiment of a medical tubing hub having recesses for retaining a magnetic member for use with a removable fastening member (substrate backing).

FIG. 5B shows an embodiment of a medical tubing hub of FIG. 5A having the magnetic member received in the recesses.

FIG. 5C shows an embodiment of a medical tubing hub of FIG. 5A having a winged plate magnetically coupled to the medical tubing hub.

FIG. 5D shows an embodiment of the winged plate of FIG. 5C.

FIG. 5E shows an embodiment of a medical tubing hub having a winged plate of FIG. 5D magnetically coupled to the medical tubing hub.

FIG. 5F includes a bird-eye view of a winged plate of FIG. 5D.

FIG. 6A shows a front view of another embodiment of a medical tubing hub.

FIG. 6B shows a bird-eye view of the medical tubing hub of FIG. 6A.

FIG. 7A shows an embodiment of a clip which can be a part of a kit with a medical tube hub.

FIG. 7B shows a side view of the clip of FIG. 7A.

FIG. 7C shows an example of a user using a medical tubing hub and the clip of FIG. 7A.

FIG. 8 shows an example wristband which can be a part of a kit with a medical tube hub.

FIG. 9 illustrates a flowchart of an example method of retaining a medical tube to a subject.

FIG. 10 illustrates an example method of removing a medical tube hub from a subject.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology includes a medical tube hub that is adapted to receive a plurality of medical tubes (e.g., IV, oxygen, other) and has a means for attaching the medical tube hub to a patient. The means for attachment can be to a medical gown or other clothing (e.g., to fabric) worn by the patient. In one embodiment, the means of attachment can be by adhesive (e.g., pressure sensitive adhesive or other medical grade adhesive) directly to the skin or clothing worn by the patient. The means of attachment can include a clip that clips the medical tube hub to fabric of clothing worn by the patient. The medical tube hub once attached to the patient (e.g., to fabric of clothing worn by the patient) can inhibit the medical tubes that are retained from moving in a manner that compromises the tubing or an insertion site of the tubing through the skin of the patient or pulls breathing tubes from their proper breathing location (e.g., from nostrils or mouth, etc.).

In one embodiment, the medical tube hub is attached to at least one medical tube and attached to the patient (e.g., attached to clothing worn by the patient) so that the medical tube hub inhibits the medical tube(s) or at least a portion of the medical tubes attached to the patient from moving relative to the patient. This is accomplished by the medical tubes being received into the hub, such as with friction fitting, to inhibit movement of the medical tubes relative to the hub. In one example, the medical tube hub can be coupled to a catheter tube and thereby inhibit the catheter tube from moving relative to the catheter tube insertion site. This lack of movement at the insertion site can inhibit bacteria or other pathogens from entering into the insertion site and infecting the patent by inhibiting movement of the catheter tube into and out of the insertion site. In another example, the medical tube hub can be coupled to an oxygen tube and thereby inhibit the oxygen tube from moving relative to the breathing location of the patient (e.g., nostril).

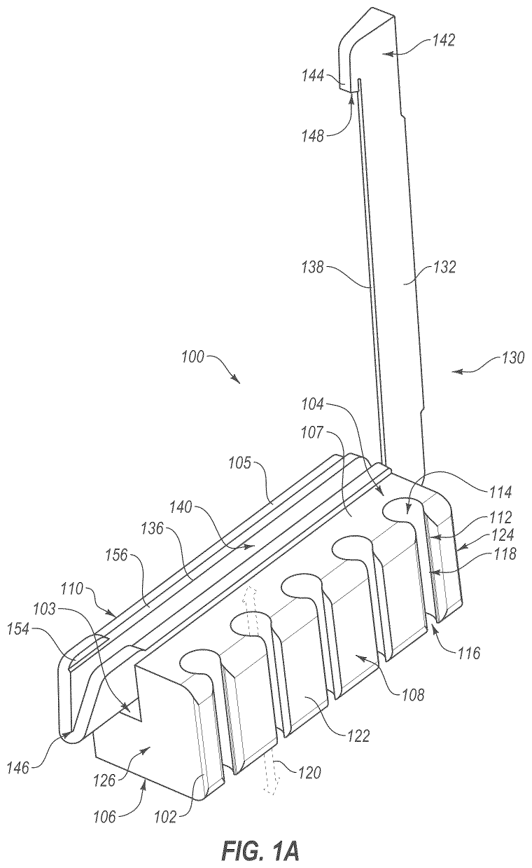

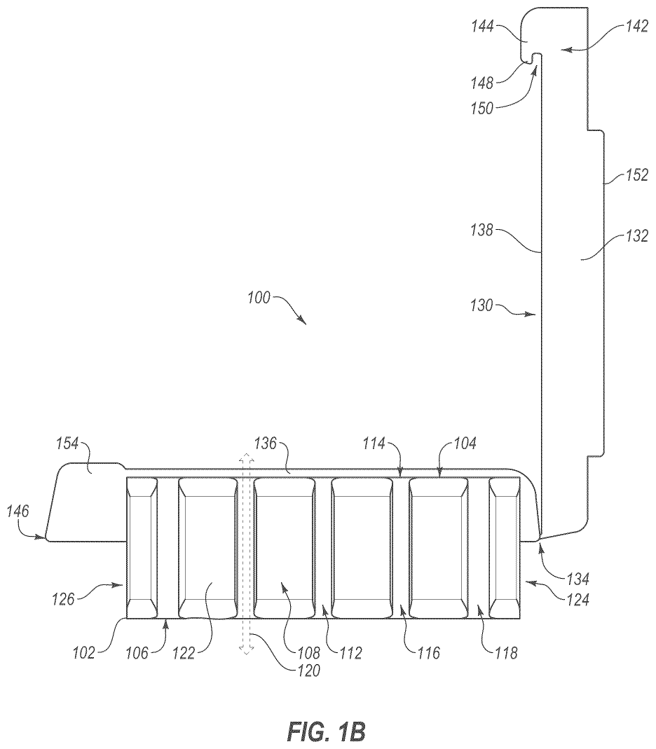

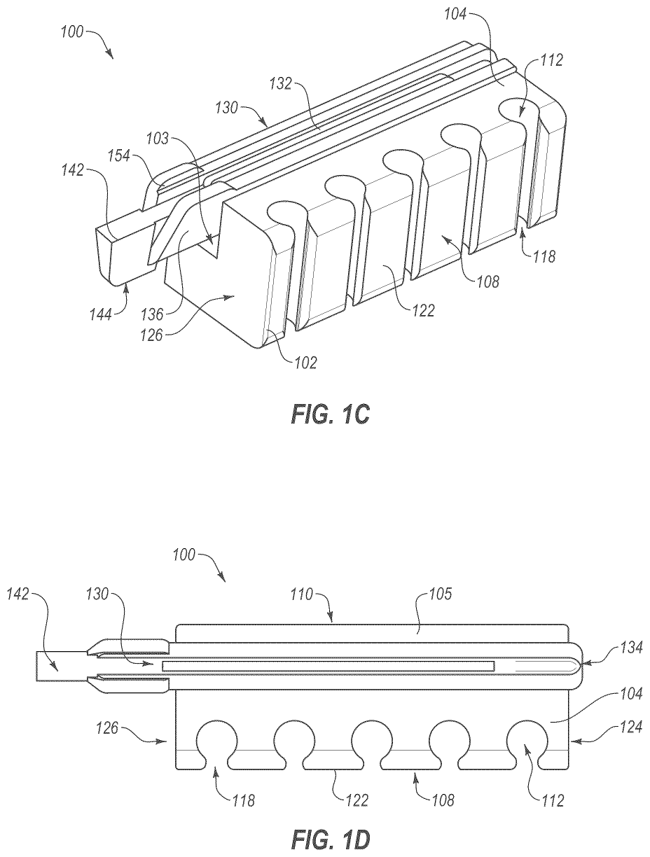

As shown in FIGS. 1A-1D, a medical tubing hub 100 can include a body 102 having a top 104, bottom 106, front 108 and back 110. The body 102 can include a plurality of conduits 112 extending from a conduit top opening 114 at the top 104 to a conduit bottom opening 116 at the bottom 106. The medical tubing hub 100 also includes an attachment means configured for attaching the body to a patient. The attachment means is shown to be a clamp, but may also be a clip (e.g., configured similar to a paper clip or alligator clip or other clip), adhesive on the back, double sided adhesive tape, safety pin, magnet(s) (e.g., when the medical tubing hub has a magnet, the patient may wear a gown or other clothing that is magnetically responsive, or the hub includes a magnetically responsive material on the back and the gown or other clothing can include the magnet), Velcro (e.g., when the medical tubing hub has one side of Velcro, such as the hooks, the other side can have the loops, or vice versa), a cinch, or other attachment devices.

The medical tube hub 100 can be configured such that each conduit 112 includes a conduit slot 118 that exposes the conduit 112 at the front 108 of the body 102. The medical tube hub 100 can include at least two conduits 112 (e.g., oxygen tube in the two conduits), or at least three conduits 112 (e.g., three IV or catheter or other tubes) or at least five conduits 112 (e.g., oxygen tube in two outer conduits and three other medical tubes) as shown. The medical tube hub 100 can include a conduit slot 118 that extends from the conduit top opening 114 at the top 104 to the conduit bottom opening 116 at the bottom 106. Each conduit 112 can have a lateral cross-sectional profile that is circular, oval, elliptical, triangular, square, rectangular, a pentagon, hexagon, heptagon, octagon, polygon, or combinations thereof, the lateral cross-sectional profile being orthogonal with a longitudinal axis 120 of the conduit 112. Each conduit 112 can have a lateral cross-sectional profile that has a conduit cross-dimension (e.g., diameter for circular conduit 112) having a first width and the conduit slot 118 has a slot cross-dimension from edge to edge that has a second width, wherein the first width is bigger than the second width, wherein the conduit cross-dimension and the slot cross-dimension edge to edge are both orthogonal to the longitudinal axis 120. As shown, the conduit 112 with the conduit slot 118 form an omega shape Ω cross-sectional profile with the front 108. The body 102 can include a plurality of conduits 112 that are parallel.

The body 102 can include a separator 122 between each conduit 112, wherein the separator 122 extends from the top 104 to the bottom 106. As shown, the body 102 can include one less separator 122 than the number of conduits 112.

The medical tube hub 100 can include the attachment means positioned and oriented between the plurality of conduits 112 and the back 110. As shown, the attachment means includes a clamp 130. However, the attachment means can be configured as any other embodiment as described herein. The clamp 130 has a lever 132 attached through a hinge 134 to a receiver 136. However, while not shown, the lever 132 can be detached from the receiver 136 as two separate pieces, which can still be used as a clamp as shown herein, such as by direct down coupling rather than rotational coupling through a hinge. As shown, the lever 132 has a latch edge 138 and the receiver 136 has a receiver slot 140, wherein the latch edge 138 and receiver slot 140 are cooperatively configured such that when in a closed position the latch edge 138 is received in the receiver slot 140. The lever 132 and receiver 136 are shown to be elongate; however, any length dimension, such as short or not elongate may also be used. When elongate, the lever 132 and receiver 136 extend between a first side 124 to a second side 126 of the body 102. Also, the lever 132 and receiver 136 may extend past the first side 124 and past the second side 126 of the body 102. The lever 132 can have a wedge shaped cross-sectional profile with a point at the latch edge 138. Correspondingly, the receiver slot 140 has a wedge shaped cross-sectional profile with a point at a trough of the receiver slot 140. However, the lever 132 may be squared or rounded, which may correspond with the shape of the receiver slot 140.

The lever 132 is shown to include a fastening head 142 opposite of the hinge 134. The fastening head 142 allows for easier coupling and decoupling of the lever 132 with the receiver 136. The fastening head 142 can include a fastening tab 144 adapted to fasten to the receiver 136. In one aspect, the receiver 136 is devoid of a notch to receive the fastening tab 144, and thereby the fastening tab 144 fastens with a fastening end 146 of the receiver 136, the fastening end 146 being opposite of the hinge 134. In one aspect, the receiver 136 includes a notch (not shown) to receive the fastening tab 144, and thereby the fastening tab 144 fastens with the notch at the fastening end 146 of the receiver 136, the fastening end 146 being opposite of the hinge 134. As shown, the fastening tab 144 is configured as a fastening hook. When the fastening tab 144 is configured as a fastening hook, the fastening hook includes a hooking member 148 such that there is a gap 150 between the hooking member 148 and the latch edge 138. Correspondingly, the fastening end 146 includes a bottom of the receiver 136 that is cooperatively dimensioned with a dimension of the gap 150, such that the bottom of the receiver 136 at the fastening end 146 fits into the gap 150.

In one embodiment, the clamp 130 is coupled with the body 102. For example, the clamp 130 is coupled with the body 102 via an adhesive. In one aspect, the clamp 130 is integrated with the body 102. For example, the clamp 130 is integrated with the body 102 so as to be a unitary body. In another example, the clamp 130 is integrated with the body 102 so as to be a unitary body made of the same material.

In one embodiment as shown, the clamp 130 is coupled with the body 102, the body 102 having a clamp recess 103 that retains the clamp 130 therein. The clamp recess 103 can be formed into the back 110 of the body 102. In some aspects, the clamp recess 103 can be formed into the back 110 and top 104 of the body 102. In some aspects, the clamp recess 103 can be formed into the top 104 of the body 102. In one option, the clamp recess 103 is formed into the top 104 of the body 102 between the back 110 and the front 108. In another option, the clamp recess 103 is formed into the top 104 of the body 102 between the back 110 and the front 108 and positioned closer to the back 110. As shown, the clamp recess 103 extends between the first end 124 and second end 126. The clamp recess 103 can be formed into the top 104 of the body 102 between the back 110 and the conduits 112.

In one embodiment, the lever 132 is longer than the receiver 136. In one aspect, the lever 132 is longer than the receiver 136 such that the fastening head 142 extends past the fastener end 146 when the clamp 130 is in a closed position. In one aspect, the lever 132 includes a grip member 152 opposite of the latch edge 138.

In one embodiment, the body 102 includes a back member 105 located at the back 110. In one aspect, the body 102 includes a back member 105 located at the back 110, the back member 105 cooperating with a main portion 107 of the body 102 to form the clamp recess 103.

In one embodiment, the fastener end 146 includes a receiver head 154. In one aspect, the fastener end 146 includes a receiver head 154 that is bigger than a main body portion 156 of the receiver 136. The receiver head 154 and fastening head 142 can be dimensionally larger to allow for coupling and decoupling thereof so that the clamp 130 can be used to clamp the hub 100 as described and shown herein. In one aspect, the clamp 132 and clamp recess 103 are orthogonal with the conduits 112, which can allow for easy clamping and insertion of medical tubes into the conduits.

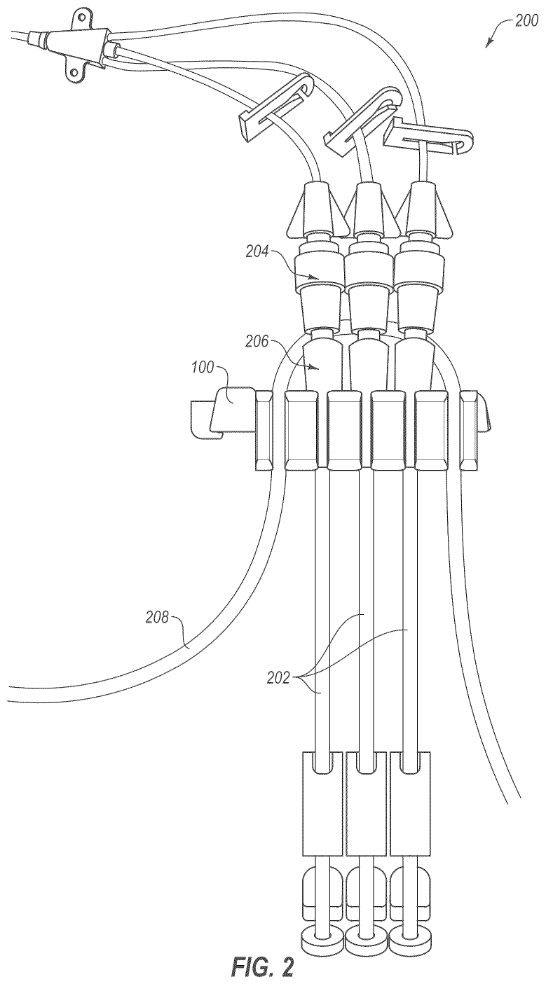

FIG. 2 shows a system 200 having the hub 100 with the medical tubes 202 located in the conduits. The medical tubes 202 can include fixtures 204 that have extensions 206 that slide down into the conduits 112. The conduits 112 retain the medical tubes 202 so that they do not slide relative to the hub 100. The material of the body of the hub 100 can be resiliently flexible, such as an elastomer or rubber-like material, to allow the conduit slots 118 to be opened up for lateral insertion into the conduits 112. However, the medical tubes 202, such as those with one end free without fixtures, can be slid longitudinally along the longitudinal axis of the conduit. The medical tubes 202 can be inserted in the conduits before or after the clamp 130 is coupled to clothing (e.g., medical gown) worn by the patient. The hub 100 can be used with only the medical tubes 202 (e.g., not with an oxygen tube 208) located in the conduits 112.

FIG. 2 also shows the system 200 with the hub 100 having an oxygen tube 208 passed through both outer conduits 112 to retain the oxygen tube 208 in the hub 100 so as to inhibit slipping or movement of the oxygen tube 208 relative to the hub 100. Passing the oxygen tube 208 up one conduit 112 and down the other conduit 112 can inhibit kinking or other harm to the oxygen tube 208. During patient movement, the hub 100 keeps the oxygen tube 208 from moving relative to the nose or mouth of the patient.

Also, the hub 100 can be used with only the oxygen tube 208 (e.g., not with the medical tubes 202) if desired, which oxygen tube 208 is passed up one conduit 112 and down another conduit 112. The conduits 112 may be both outer conduits 112, however, depending on tube flexibility the oxygen tube 208 may be located in any suitable conduit 112. Also, it may be possible to use only one conduit 112 receiving the oxygen tube 208.

A method of use can include the medical tube hub 100 being attached via the clamp 130 to the clothing worn by the patient. While the clothing can be a medical gown, the clothing can be any shirt, jacket, gown, or other clothing. For attachment, a portion of the fabric of the clothing is pulled so that the clamp 130 can fit and fasten therearound. While the hub 100 can be attached to be substantially horizontal, it may also be at any angle such as vertical or upside down. The positioning of the hub 100 can retain the medical tubes 202 and oxygen tubes 208 without movement so that the medical tube 202 (e.g., catheter tube) does not move or slide relative to the insertion site into the patent when the medical tube 202 is inserted into a patient or connected into an inserted member. This use of the hub 100 allows the patient to move freely without risking dislodging the catheter or other medical tube 202 relative to the insertion site. As such, patients using the hub 100 may have more freedom of movement and can be allowed to perform more activities while having a catheter or other medical tube inserted into their body. The same principle applies to the oxygen tube 208, where the hub 100 inhibits the oxygen tube to be pulled from them if the other end gets snagged.

Additionally, use of an oxygen tube 208 by a patient can be improved without snagging to pull the oxygen tube 208 from the face/nose of the patient by being retained by the hub 100. Now, additionally the hub 100 inhibits the oxygen tube 208 from being movable relative to the patient (e.g., relative to the nose). Accordingly, the catheter tubes or other medical tubes 202 are in the central conduits 112, and the oxygen tube 208 is located in the outer conduits 112. This use of the hub 100 allows the patient to move freely without risking dislodging the catheter or other medical tube 202 relative to the insertion site in the body or disturbing the oxygen tube 208 relative to the face. As such, patients using the hub 100 may have more freedom of movement and can be allowed to perform more activities while having a catheter or other medical tube 202 inserted and having an oxygen tube 208 for breathing.

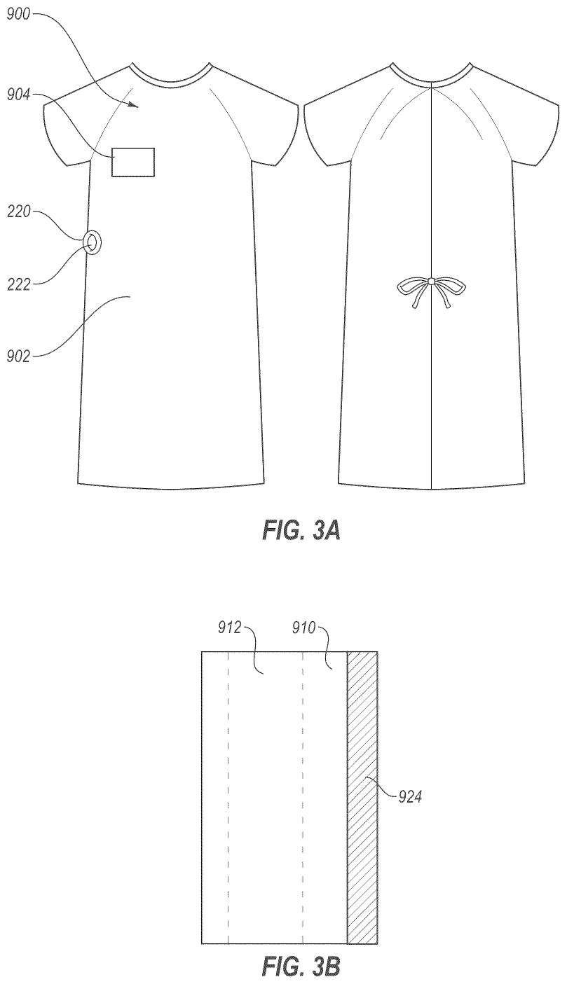

FIG. 3A shows the front (left) and back (right) of a medical gown 900 adapted to couple with attachment means of the medical tube hub. The medical gown 900 is shown to include fabric 902 and optionally an attachment member 904. The attachment member 904 can be a part of the hub, such as the clamp or a magnetically responsive member (e.g., magnet or metal) that associates with the hub. Alternatively, the attachment member may be coupled to the gown 900 or integrated therewith. As shown, the attachment member 904 is rectangular, but it can be any shape or size. As shown, the attachment member 904 is on the outside of the fabric 902 on the patient's right side; however, the attachment member 904 could be on the inside of the fabric 902 if feasible, and it may be at any location (for example on the patient's left side). Moreover, the medical gown 900 can include a plurality of attachment members 904 at any locations relative to the fabric 902. The attachment member 904 can be adapted in various configurations for coupling with the medical tube hub. When the medical tube hub includes a clamp or clip or safety pin as the attachment means, the attachment member 904 can be a reinforced member that has more structural strength than the fabric 902. When the attachment means is adhesive on the back of the hub, the attachment member 904 is a substrate that can receive the adhesive. When the attachment means is a magnet the attachment member 904 is magnetically responsive material, or the hub is magnetically responsive and the attachment member 904 is the magnet. The attachment means and attachment member 904 may cooperatively be Velcro (e.g., when one has one side of Velcro, such as the hooks, the other can have the loops, or vice versa).

Additionally, FIG. 3A shows that a kit having the hub can also include a tube clamp 220 that has an aperture 222 for receiving a medical tube or oxygen tube therethrough. The clamp 220 can be any type of clamp that can clamp onto the clothing such as the medical gown 900 or shirt to provide the aperture 222 adapted to receive the medical tube or oxygen tube there through. The clamp 220 can be especially beneficial to hold a part of the oxygen tube during movement or sleep so that the oxygen tube stays fastened to the patient in the proper position. For example, the oxygen tube can be applied to the face of a patient, then pass through the one or two conduits of the hub, and then down through the clamp 220. This makes walking or doing other activities, even sleeping, better without the risk of pulling the oxygen tube from the face of the patient. The clamp 220 cooperates with the hub for securing the oxygen tube or other medical tube to the patient.

FIG. 3B shows a side view of a medical device hub 910 that has attachment means 924 that attaches to the attachment member 904. Conduits 912 are shown with dashed lines. As shown, the medical device hub 910 can be low profile from front to back, meaning the body is taller from top to bottom than it is thick from front to back. The lateral side length from right to left can vary. The low profile body can be beneficial for the fastening means, including the clip, clamp, magnetic, Velcro, adhesive, or others.

In some embodiments, the medical tube hub can be used for only an oxygen tube, which is passed through the outer conduits. When the hub includes a clothing clamp, which can also be used with the hub (e.g., and provided with the hub as a kit), the clothing clamp can keep a distal end of the oxygen tubing from moving, which cooperates with the medical tube hub for inhibiting movement or sliding of the oxygen tube relative to the patient. As described earlier, this allows for significantly more patient movement with a lower risk of catching or snagging the oxygen tube. Also, the medical tube hub can retain the medical tubes so that the patient does not trip over the tubes, thereby overcoming a common problem of patients tripping over long oxygen tubes.

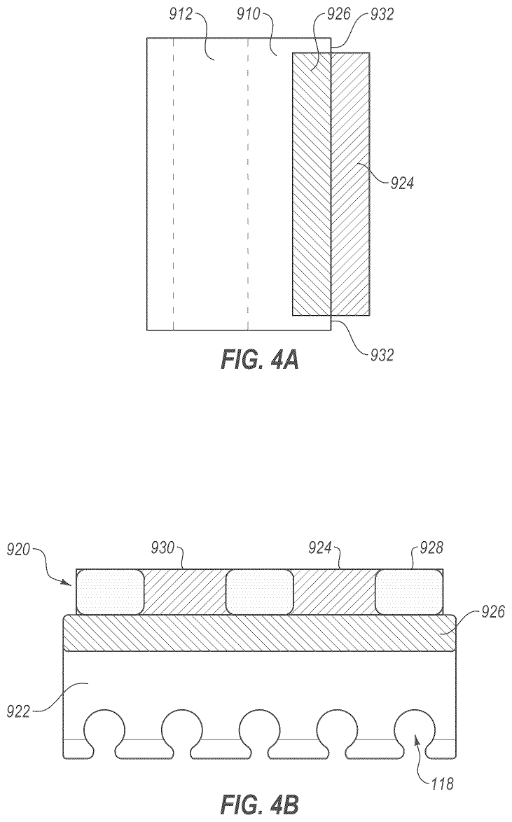

FIGS. 4A-4B show another embodiment of a medical tube hub 920 that includes the body 922 having a hub magnetic member 926 that can magnetically couple with at least one removable magnetic fastener 924, and optionally at least two removable magnetic fasteners 924. The hub magnetic member 926 can be integrated, coupled or otherwise permanently associated with the body 922 of the hub 920. The removable magnetic fastener 924 may include one or more magnetic members 928, which can be magnetic or magnetically responsive that can magnetically interact with the hub magnetic member 926. When including more than one magnetic member 928, the removable magnetic fastener 924 can include a fastener substrate 930 that retains the magnetic members 928. However, it should be recognized that the fastener substrate 930 may include only one magnetic member 928 and still work. In some instances, a single magnet or magnetically responsive member can be used for the removable magnetic fastener 924, with or without a fastener substrate 930. As shown, there are magnetic members 928 at different locations. However, only one magnetic member 928, which can be in the middle may be used. Alternatively, two magnetic members 928 spaced apart on the back can be used, or on the back toward the ends may be used. Similarly, the hub magnetic member 926 can include at least one magnetically responsive material or magnetic material or a plurality thereof, which can be similar to the magnets or magnetically responsive members 928 relative to the fastener substrate 930. During use, the hub 920 can be placed on an article of clothing, such as a medical gown or shirt with the first magnetic member 926 contacting the article of clothing, and then the removable magnetic fastener 924 can be placed on the other side of the fabric of the clothing and magnetically associated with the first magnetic member 926 in order to hold the hub 920 to the clothing via magnetic coupling. The magnetic or magnetically responsive materials of the first magnetic member 926 and/or magnetic fastener 924 (e.g., magnetic member 928) can have various magnetic strengths so long as the hub is capable of being magnetically held to the clothing without moving via the removable magnetic fastener 924.

While the first magnetic member 926 and/or removable magnetic fastener 924 may extend from the top to the bottom and/or the right side to the left side, the first magnetic member 928 and/or removable magnetic fastener 924 may have smaller relative dimensions to the body 922. Also, the body 922 can include a lip 932 at the top, bottom, right, or left sides that overhangs and allows the removable magnetic fastener 924 to magnetically couple with the first magnetic member 926 by a normal movement vector, but prevents sliding of the removable magnetic fastener 924 relative to the first magnetic member 928. As known, magnetics can have strong magnetic attraction when pulled normal to the surface of the magnet, but the same magnet may allow sliding. The lip 932 inhibits sliding of the removable magnetic fastener 924 relative to the first magnetic member 926. The body 922 can include one lip 932 (e.g., at top) or a plurality of lips 932.

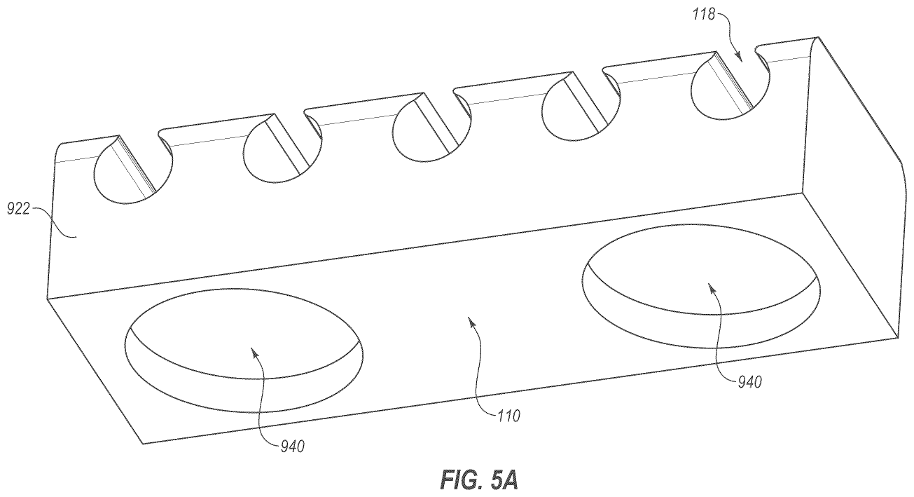

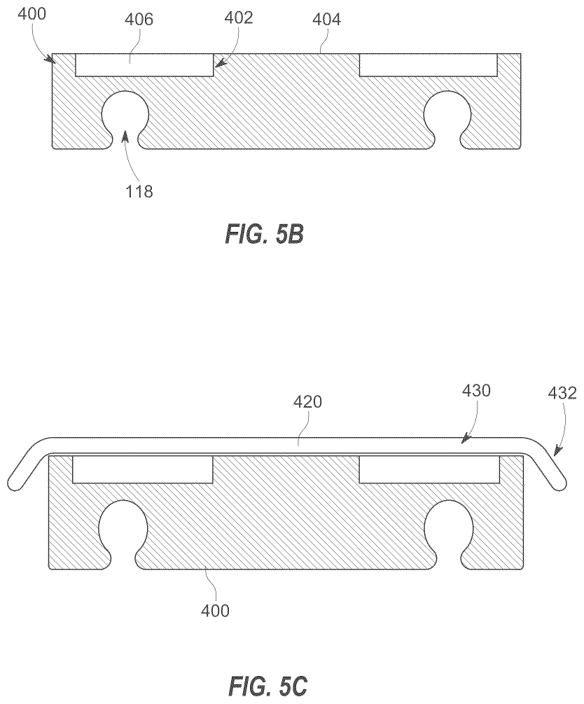

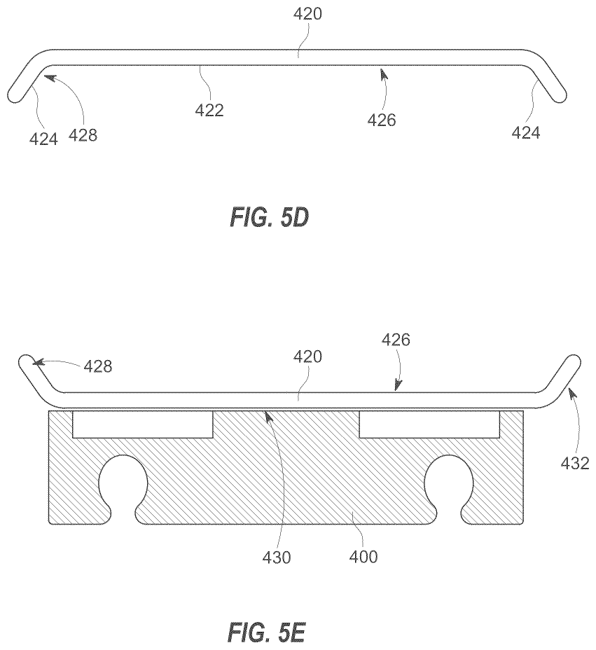

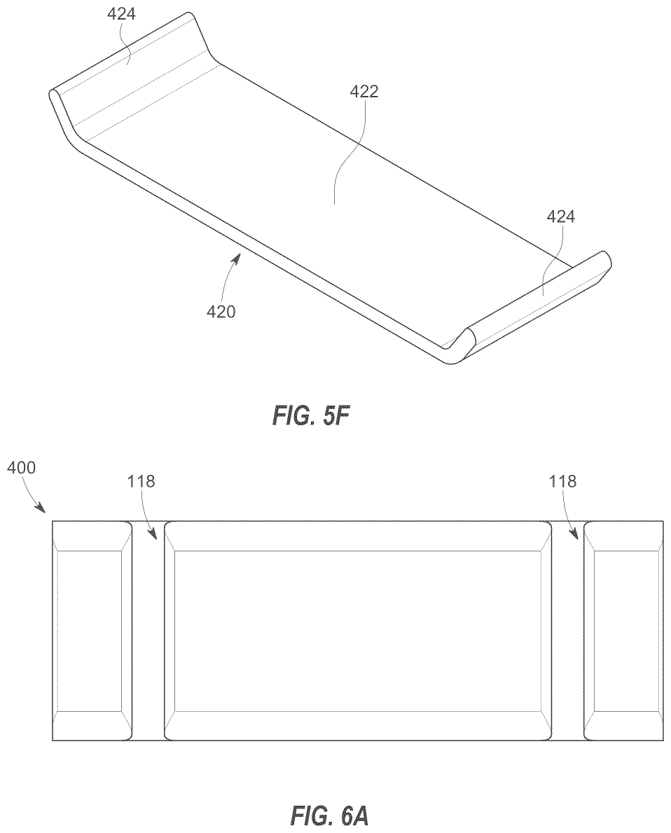

FIG. 5A shows another embodiment of the medical tube hub 400 that has recesses 402 on the back side 404. In some embodiments, the recesses 402 may be configured to receive magnetic members (not shown) therein. In these and other embodiments, the recesses 402 and the magnetic members may be shaped substantially similar such that the magnetic members may fit into the recesses 402. For example, the recesses 402 and the magnetic members can be substantially circular. In other embodiments, the recesses 402 and the magnetic members may have any other suitable shapes, such rectangle, square, triangle, among others. In some embodiments, the medical tube hub 400 may include any number of the recesses. For example, although Figures may illustrate the medical tube hub 400 as having two recesses 402, the medical tube hub 400 may have other numbers of the recesses 402, such as one recess, three recesses, 4 recesses, 5 recesses, etc. In some embodiments, each recess 402 may receive a magnetic member therein. In other embodiments, certain number of the recesses 402 may receive magnetic members therein. The magnetic member(s) can then magnetically couple with a magnetically responsive back plate (not shown). The conduit slots 118 for medical tubes (5 are shown) are also illustrated. The medical tube hub 400 may include any other suitable number of conduit slots.

FIG. 5B shows another embodiment of the medical tube hub 400 having 2 tubing conduits 118 with a magnet 406 in each of the recesses 402 on the back side 404. In some embodiments, the magnet 406 may be received by the recesses 402 such that the magnet 406 fits snuggly in the recesses 402. For instance, the magnet 406 may fit in the recesses 402 such that the magnet 406 substantially remains in the recesses 402. Additionally or alternatively, the magnet 406 may be fixed in the recesses 402 via an adhesive.

FIG. 5C shows the medical tube hub 400 having a winged plate 420 (e.g., stabilizer bar) magnetically coupled to the magnets 406 in order to be magnetically coupled to the back side 404. The winged plate 420 can include a base portion 422 and a wing 424 extending from opposite sides of the base portion 422. FIG. 5C shows the winged plate 420 in the tight magnetic coupling orientation where the back side 404 of the medical tube hub 400 is touching the base portion 422 of the winged plate 420 such that the wings 424 are pointed upwardly and toward the medical tube hub 400. This creates a fixing surface 426 between the two angled wing surfaces 428 that are oriented closely together and oriented in the same direction. On the other side of the winged plate 420 are the sliding surface 430 and outer wing surfaces 432. When the back side 404 of the medical tube hub 400 with the magnets 406 are magnetically coupled with the fixing surface 426, the angled wing surfaces 428 form a narrow gap with the sidewalls of the hub 400, which makes it difficult to pull the winged plate 420 off of the hub 400. The small gap is triangled shape, which allows a fingertip to be pressed into it to help facilitate pulling the winged plate 420 off the hub 400.

Additionally, the magnets 406 can be selected so that they have sufficient strength so that it is difficult for the winged plate 420 to be pulled from the hub 400. For instance, the magnets 406 may be strong enough such that the hub may remain attached to the winged plate and the subject without being removed as the subject is moving. The winged plate 420 includes a magnetically responsive material that is highly magnetic with the magnets. Such magnetic nature may create a coupling between the winged plate 420 and the hub 400 that does not come apart unless specifically separated (e.g., pried apart). This prevents accidental decoupling and having the hub fall from the patent to jeopardize the medical tubes. The winged plate 420 may be made of any suitable materials such as magnetically responsive metals. For example, the winged plate 420 may be made of steel and alloys thereof are specific examples.

FIG. 5D shows the winged plate 420 can include a base portion 422 and a wing 424 extending from opposite sides of the base portion 422.

FIG. 5E shows the winged plate 420 in a loose magnetic coupling position with respect to the hub 400. Here, the sliding surface 430 is facing the magnets 406 so that the fixing surface 426 is pointing away from the magnets 406/hub 400. The wings 424 point away from the hub 400, which provides handles for twisting the winged plate 420 relative to the hub 400, which allows for separation. Here, the loose magnetic coupling is not recommended for holding the hub 400 to a patient. For instances, the wings 424 may be pressed against a body of the patient such that it may be uncomfortable for the patient to wear the hub 400. However, loose magnetic coupling position can be used for testing locations of mounting the hub on the patient's garments.

Once a suitable position is found that is favorable to the patient, the back surface 404 of the hub 400 is mounted onto the fixing surface 426 so that the hub 400 is between the angled wing surfaces 428. This fixed position is shown in FIG. 5C, which is used for the coupling of the hub 400 to the patient's garment by the winged hub 420 being on the opposite side of the garment to use magnetic force to hold the garment between the hub 400 and the winged plate 420. FIG. 4A shows such a garment, where the winged plate 420 can be on the inside with the hub 400 on the outside so that the conduits 118 can be used for medical tubes.

FIG. 5F shows another view of the winged plate 420 of FIG. 5D. The winged plate 420 may include a base 422 and wings 424 extending upward from the base 422. The angle formed by the wings 424 relative to the base 422 may also vary, such as from 170 degrees to about 105 degrees, or from 150 degrees to 110 degrees or about 140 degrees to about 120 degrees, or about 125 degrees.

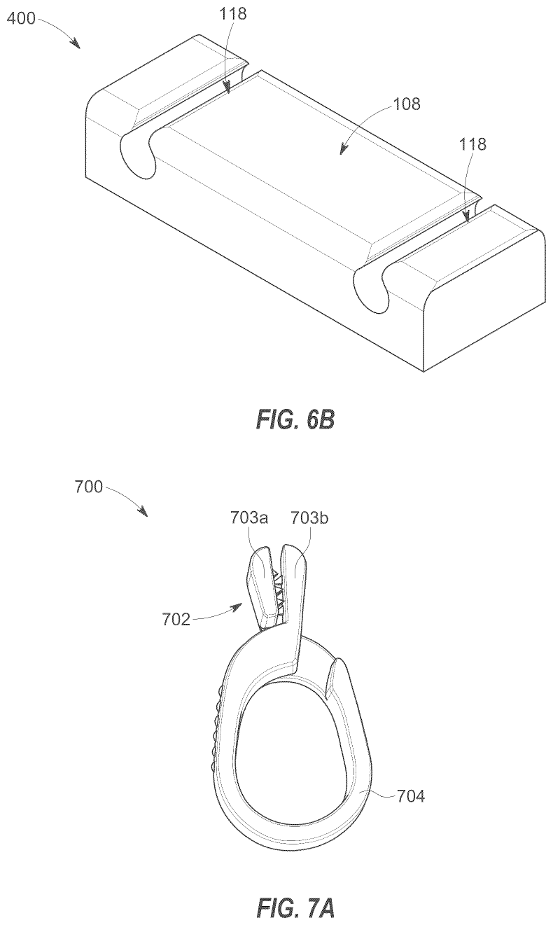

FIG. 6A shows a front view of another embodiment of a medical tubing hub 400. The hub 400 may include one or more conduits 118 configured to receive one or more medical tubes therein. The hub 400 may include different numbers of the conduits 118. For example, the hub 400 may include 1, 2, 3, 4, or 5 conduits. FIG. 6A shows a bottom view of the hub 400.

FIG. 6B shows a bird-eye view of the medical tubing hub 400 of FIG. 6A. The hub 400 includes one or more conduits 118 on front side 108 of the hub 400. The front side 108 may be the side facing away from a subject using the hub 400. FIG. 6B may illustrate shapes of the one or more conduits 118. For example, the conduits 118 may be substantially circular to correspond to shapes of medical tubes received therein. While illustrated as being substantially circular, the conduits 118 can have any other suitable shapes.

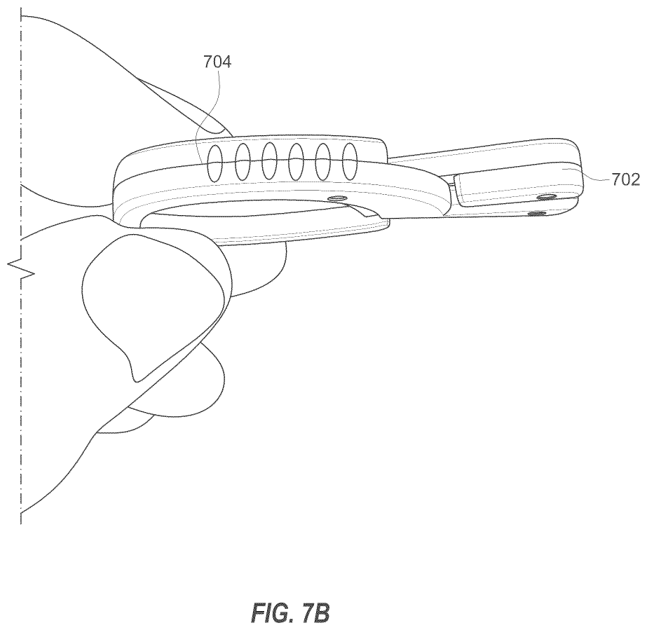

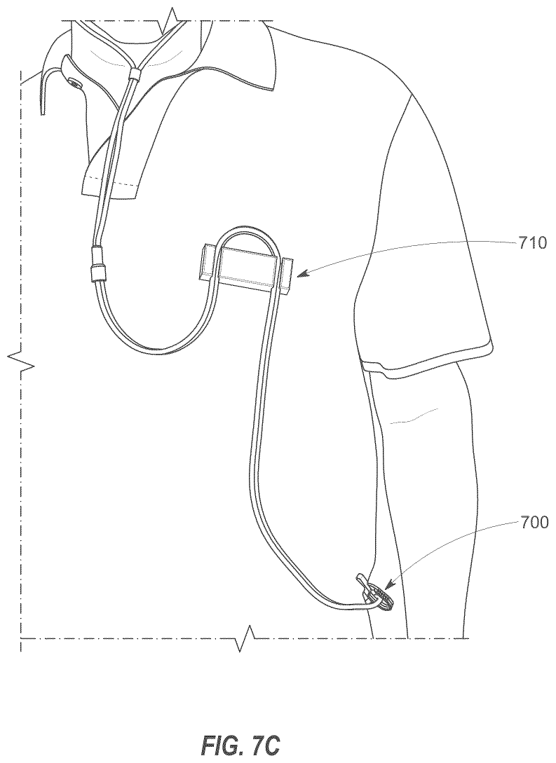

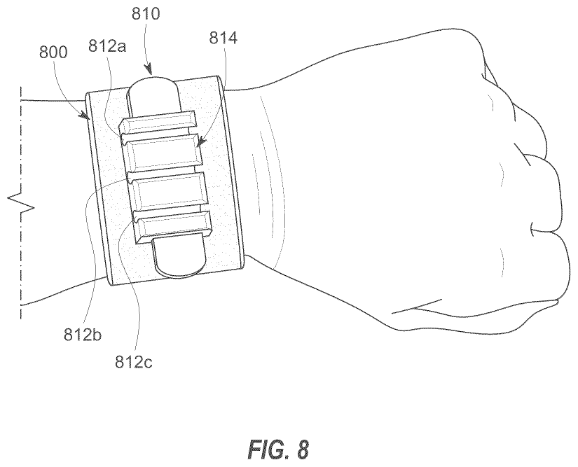

FIGS. 7A-7B show an example of a clip 700 according to one or more embodiments of the present disclosure. In some embodiments, the clip 700 may be a part of a kit with the medical tubing hub such as illustrated with respect to FIGS. 6A-6B. For example, the clip 700 may be used with the hub to further retain and direct medical tubes. In some embodiments, the clip 700 may include a first portion 702 and a second portion 704. The first portion may include a clipping mechanism configured to attach the clip 700 to an item such as a clothing item. The first portion may include a first clip leg 703a and a second clip leg 703b. A clothing item may be placed between the first clip leg 703a and the second clip leg 703b such that the clip 700 is attached to the clothing item. In some embodiments, the first clip leg 703a and the second clip leg 703b may be separated by applying pressure to the second portion 704 and may be closed (e.g., the first clip leg 703a and the second clip leg 703 meet each other) by releasing the applied pressure to the second portion 704.

In these and other embodiments, the second portion 704 may be configured as a handle to open and close the first portion 702. Additionally or alternatively, the second portion 704 may be configured to hold a medical tube. For example, the second portion 704 may include a loop such that that a medical tube may be placed through the loop. The loop may hold the medical tube at the level the clip 700 is placed on. Additionally, the loop may be configured to guide the medical tube to a certain direction. In some embodiments, the clip 700 may be made of sufficiently rigid material such that the clip 700 may be securely attached to clothing items while holding medical tubes. For example, the clip 700 may be made of steel, aluminum, plastic, among others.

FIG. 7C shows the clip 700 attached to a user's clothing to the side. For example, the clip 700 is illustrated as being coupled to the side of the user's t-shirt. The clip 700 may be coupled to other parts or other types of clothing items. The clip 700 may receive a medical tube therein through the loop from a medical tube hub 710. For example, the medical tube may be used to deliver oxygen to the user. The medical tube may be attached to the medical tube hub 710 such that the medical tube is held in place and not moving around, while maintaining flow of oxygen. The medical tube may be further received by the clip 700 such that the medical tube is guided to side and/or back of the user. Such guidance of the medical tube may improve the user's mobility with respect to the medical tube as the medical tube is moved to reduce the risk of the user tripping over the medical tube or the medical tube catching on objects, such as handles, door knobs, etc. as the user is moving. Additionally or alternatively, the clip 700 may hold and/or guide the medical tube at a certain location or a direction such that the medical tube is not moving around to interfere with the user's activities. For example, the clip 600 may guide the medical tube to the side or back of the user while the user is laying down such that the medical tube is out of the user's way.

FIG. 8 shows an example wristband 800, according to one or more embodiments of the present disclosure. In some embodiments, the wrist band 800 may be a part of a kit with the medical tubing hub such as illustrated with respect FIGS. 1A, 5A and/or 6A-6B. In some embodiments, the wrist band 800 may include a tube holder 810 including one or more conduits. For example, the tube holder 810 may be attached on the wrist band 800. In some embodiments, the tube holder 810 may include a first conduit 812a, a second conduit 812b, and a third conduit 812c (collectively referred to as the “conduits 812”). While illustrated as having three conduits 812, the tube holder 810 may include any suitable number of conduits. For example, the tube holder may include 1, 2, 3, 4, or 5 conduits therein. In some embodiments, the conduits 812 may be separated by a separator 814 placed between each of the conduits 812. In some embodiments, the conduits 812 may be configured to receive one or more medical tubes therein. In some embodiments, the material of the tube holder 810 can be resiliently flexible, such as an elastomer or rubber-like material. In some embodiments, the conduits 812 may be similar to the conduits 112 of the medical tube hub 100.

In some embodiments, the wrist band 800 may be made of elastic materials such that the wrist band 800 may stay in position with respect to the user wearing the wrist band 800. For example, the wrist band 800 may be made of cotton, nylon, polyester, spandex, Lycra, elastane, or a combination of one or more materials. The wrist band 800 may retain one or more medical tubes within the tube holder 810 such that the medical tubes may remain attached to the user's hand and/or wrist. For example, the medical tubes may move along with the user's hand wearing the wrist band 800, such that the medical tubes may not interfere with the user's movements (e.g., entangle with the user's hands or arms).

In some embodiments, the tube holder 810 may be removably attached to the wrist band 800. For example, the wrist band 800 and the tube holder 810 may be coupled using hook-and-loop fasteners. For example, the wrist band 800 and the tube holder 810 may be covered with and/or made of materials that may couple with each other, such that the tube holder 810 may be removably attached to the wrist band 800. In some embodiments, the tube holder 810 may be removably attached to different parts of the wrist band 800. For example, the wrist band 800 may be covered and/or made of similar materials throughout such that the tube holder 810 may attach to any part of the wrist band 800.

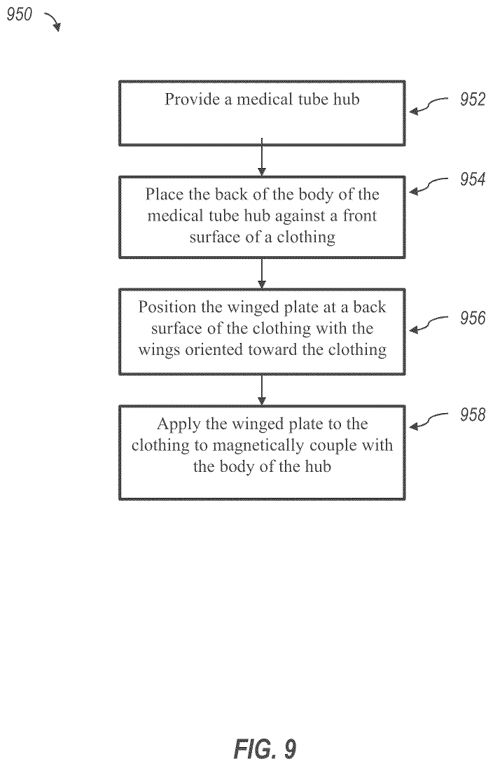

FIG. 9 illustrates a flowchart of an example method 950 of retaining a medical tube to a subject, in accordance with one or more embodiments of the present disclosure. Although illustrated with discrete blocks, the steps and operations associated with one or more blocks of the method 950 may be divided into additional blocks, combined into fewer blocks, or eliminated, depending on the particular implementation.

The method 950 may include block 952. At block 952, a medical tube hub may be provided. In some embodiments, the medical tube hub may be the medical tube hub 400 of FIGS. 5A and/or 6A.

At block 954, back of the body of the medical tube hub may be placed against a front surface of a clothing. The clothing may include a clothing worn by the subject, such as a t-shirt, gown, shirt, among others. In some embodiments, the medical tube hub may be placed at a location convenient for the subject. For example, the hub may be placed at chest level of the subject.

At block 956, the winged plate may be positioned at a back surface of the clothing with the wings oriented toward the clothing. In some embodiments, the winged plate may be located such that the body of the hub and the winged plate may be substantially aligned with each other. For example, the body and the plate may be placed to mate with each other with the clothing in between the body and the plate. In some embodiments, the winged plate may be positioned with the wings oriented away from the clothing.

At block 958, the winged plate may be applied to the clothing to magnetically couple with the body of the hub. In some embodiments, the body of the hub may include one or more magnets configured to magnetically mate with the winged plate. In these and other embodiments, the one or more magnets may be sufficiently strong to provide a large magnetic coupling that is difficult to remove.

Modification, additions, or omissions may be made to the method 900 without departing from the scope of the present disclosure. For example, the outlined steps and operations are only provided as examples, and some of the steps and operations may be optional, combined into fewer steps and operations, or expanded into additional steps and operations without detracting from the essence of the disclosed embodiments.

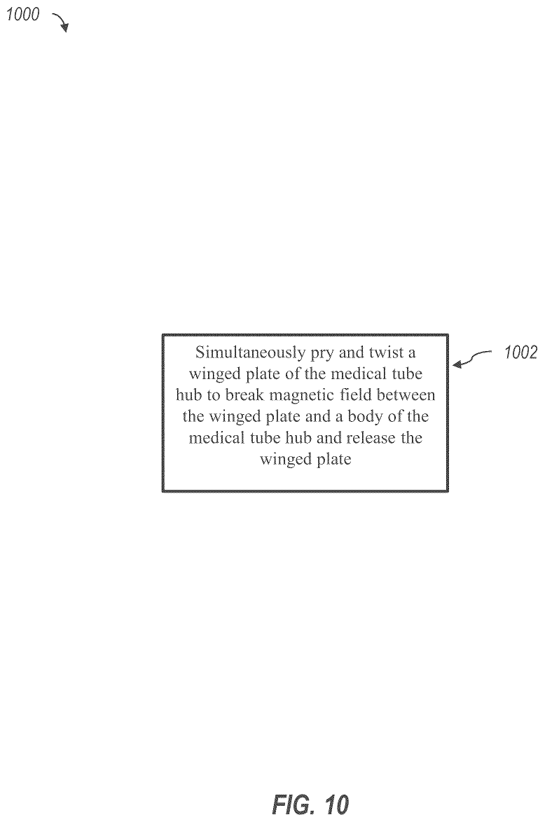

FIG. 10 illustrates an example method 1000 of removing a medical tube hub from a subject, in accordance with one or more embodiments of the present disclosure. Although illustrated with discrete blocks, the steps and operations associated with one or more blocks of the method 1000 may be divided into additional blocks, combined into fewer blocks, or eliminated, depending on the particular implementation.

The method 1000 may include block 1002. At block 1002, a winged plate of the medical tube hub may be pried and/or twisted to break magnetic field between the winged plate and a body of the medical tube hub. In response to the magnetic field being broken the winged plate may be released from the body of the medical tube hub. In some embodiments, the winged plate may be pried and twisted simultaneously such that the magnetic field may be broken.

Modification, additions, or omissions may be made to the method 1000 without departing from the scope of the present disclosure. For example, the outlined steps and operations are only provided as examples, and some of the steps and operations may be optional, combined into fewer steps and operations, or expanded into additional steps and operations without detracting from the essence of the disclosed embodiments.

For example, the method 1000 may further include rotating the winged plate relative to the medical tube hub from being aligned with the medical tube hub to be at an angle, such as perpendicular therewith, where now only one magnet of the medical tube hub magnetically holds the winged plate with a magnetic field, wherein at least one magnetic field is broken between the medical tub hub and the winged plate. Additionally, the winged plate may be slid from the medical tube hub to break the magnetic field.

In some embodiments, the medical tube hub may be rotated relative to the winged plat from being aligned with the winged plate to be at an angle, such as perpendicular therewith, where now only one magnet of the medical tube hub magnetically holds the winged plate with a magnetic field, wherein at least one magnetic field is broken between the medical tub hub and the winged plate. Additionally, the winged plate may be slid from the medical tube hub to break the magnetic field.

In some embodiments, a method includes: positioning the medical tube hub on a garment surface; positioning the winged plate in a loose magnetic coupling position with the wings pointed away from the hub; removing the winged plate from being magnetically coupled with hub; repositioning the medical tub hub on the garment surface; and positioning the winged plate in a tight magnetic coupling position with the wings pointed toward the hub.

The magnetic can be a sufficiently strong magnet that provides a large magnetic coupling that is difficult to remove. The metal winged plate can be ST-G90 galvanized steel.

In some embodiments, the magnets can be neodymium magnets or other strong magnet with a pull rating of at least 2.5 lbs of pull, more preferably at least 4 lbs of pull, more preferably at least 5 lbs of pull, and most preferably at least 7 lbs of pull. The higher the pull the stronger the fixing magnetic coupling.

The shape of the magnet can vary and can be a disk as shown. However, other shapes can work for the recess and the magnet fixed therein (e.g., adhesive).

The body of the hub can also be made of various materials. An elastomeric material allows for the body to flex to open the conduits to receive the tubs and then resiliently return back to the original shape to retain the tubes in the conduits. An example material includes thermoplastic polyurethane (TPU). In certain blends it can become very soft, but TPU material offers many benefits and features. 3D printed parts with TPU are durable and have the ability to withstand ambient temperatures of up to 80 degrees Celsius.

TPU filament is abrasion resistant, can withstand impacts and is resistant to many chemicals. Its versatile and used in many different industries. There are different versions of TPU material but in the main it can be classed as two types. Firstly known as Polyether Polyurethane and secondly Polyester Polyurethane. With both having different characteristics that can be suited to a specific need. PU (Thermoplastic polyurethane) is an elastomeric copolymer, made up of alternating soft and hard blocks, which give hardness and flexibility at the same time. This material is ideal for those applications where flexibility and durability are highly desired. With Shore-A hardness of 98, TPU is a resistant material for several industrial applications, both mechanical or chemical. Moreover, it's been especially designed to improve the 3D printing experience.

For example, the properties of the TPU can be: elongation of 450%; tensile strength of 150 MPa; shore hardness of 98 A; melting temperature of 225 degrees C.; and a glass transition temperature of 60 degrees C.

For the methods disclosed herein, the operations performed in the processes and methods may be implemented in differing order. Furthermore, the outlined operations are only provided as examples, and some operations may be optional, combined into fewer operations, eliminated, supplemented with further operations, or expanded into additional operations, without detracting from the essence of the disclosed embodiments.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled. The terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A medical tube hub comprising:
a body having a top, bottom, front, and back, the body comprising:
a plurality of conduits on the front extending from a conduit top opening at the top to a conduit bottom opening at the bottom;
a plurality of recesses in the back; and
a plurality of magnets in the plurality of recesses, wherein each magnet includes a pull strength of at least 2 pounds of pull; and
a winged plate having a base portion with wing portions extending therefrom at an angle, wherein the base portion is dimensioned about the same as the back of the body of the hub, wherein the winged plate is a magnetically responsive material and is configured to be magnetically coupled with the body of the hub.

2. The medical tube hub of claim 1, wherein each conduit includes a conduit slot that exposes the conduit at the front of the body.

3. The medical tube hub of claim 1, comprising two conduits.

4. The medical tube hub of claim 2, wherein each conduit has a lateral cross-sectional profile that has a conduit cross-dimension having a first width and the conduit slot for each conduit has a slot cross-dimension from edge to edge that has a second width, wherein the first width is bigger than the second width, wherein the conduit cross-dimension and the slot cross-dimension edge to edge are both orthogonal to a longitudinal axis.

5. The medical tube hub of claim 1, comprising a separator between each conduit, wherein the separator extends from the top to the bottom.

6. The medical tube hub of claim 1, wherein each magnet is a neodymium magnet.

7. The medical tube hub of claim 6, wherein each neodymium magnet is a disc magnet with a pull strength of at least 5 pounds of pull.

8. The medical tube hub of claim 7, wherein each neodymium magnet is a disc magnet with a pull strength of at least 7 pounds of pull.

9. The medical tube hub of claim 6, wherein the magnet magnetically couples the winged plate with the body of the hub with a clothing member between the hub and the winged plate.

10. The medical tube hub of claim 1, wherein the magnets are disc magnets.

11. The medical tube hub of claim 10, wherein the magnets include an exposed magnet surface in the body of the hub.

12. The medical tube hub of claim 1, wherein each wing is at an angle from 170 degrees to 105 degrees from the base of the winged plate.

13. The medical tube hub of claim 12, wherein the angle is between 120 degrees and 130 degrees.

14. A kit comprising:
the medical tube hub of claim 1; and
a clip having a clipping portion and a loop portion, wherein the clipping portion is configured to attach to a clothing item and the loop portion is configured to receive a medical tube.

15. The kit of claim 14, wherein the medical tube received by the loop portion is directed toward sides or back of a subject using the medical tube.

16. A kit comprising:
the medical tube hub of claim 1; and
a wrist band, the wrist band comprising one or more conduits configured to receive a medical tube therein.

17. A method of retaining a medical tube to a subject, the method comprising:
providing the medical tube hub of claim 1;
placing the back of the body of the medical tube hub against a front surface of a clothing;
positioning the winged plate at a back surface of the clothing with the wings oriented toward the clothing; and
applying the winged plate to the clothing to magnetically couple with the body of the hub.

18. A method of removing a medical tube hub from a subject, comprising:
simultaneously prying and twisting a winged plate of the medical tube hub to break magnetic field between the winged plate and a body of the medical tube hub and release the winged plate.

19. The method of removing the medical tube hub from the subject of claim 18, further, comprising:
rotating the winged plate relative to the medical tube hub from being aligned with the medical tube hub to be at an angle, such as perpendicular therewith, where now only one magnet of the medical tube hub magnetically holds the winged plate with a magnetic field, wherein at least one magnetic field is broken between the medical tub hub and the winged plate; and
sliding the winged plate from the medical tube hub to break the magnetic field.

20. The method of removing the medical tube hub from the subject of claim 18, further, comprising:
rotating the medical tube hub relative to the winged plate from being aligned with the winged plate to be at an angle, such as perpendicular therewith, where now only one magnet of the medical tube hub magnetically holds the winged plate with a magnetic field, wherein at least one magnetic field is broken between the medical tub hub and the winged plate; and
sliding the winged plate from the medical tube hub to break the magnetic field.
